(19) United States
(12) Patent Application Publication
(10) Pub. No.: US 2012/0189987 A1
(43) Pub. Date: Jul. 26, 2012

(54) SIMPLIFIED CHEMICALLY BONDED CERAMIC BIOMATERIAL COMPRISING TWO BINDER SYSTEMS

(75) Inventors: Leif Hermansson, Molle (SE); Jesper Lööf, Balinge (SE); Adam Faris, Uppsala (SE)

(73) Assignee: DOXA AB, Uppsala (SE)

(21) Appl. No.: 13/499,449

(22) PCT Filed: Oct. 9, 2009
(86) PCT No.: PCT/SE2009/051135

§ 371 Date: Mar. 30, 2012

(51) Int. Cl. A61C 5/11; A61K 6/00; B32B 37/14; A61K 6/083

(52) U.S. Cl. 433/228.1; 523/116; 523/118; 156/326; 156/332

(57) ABSTRACT

The present invention generally relates to chemically bonded ceramic biomaterials, preferably a dental material or an implant material, comprising two binder systems. The main binder system forms a chemically bonded ceramic upon hydration thereof, and comprises powdered calcium aluminate and, optionally, minor amounts of calcium silicate. The second binder system is a cross-linking organic binder system which provides for initial crosslinking of the freshly mixed paste forming the biomaterial. The biomaterial also comprises inert filler particles. The inventive biomaterial is free from reactive glass and thus provides for a simplified two binder systems biomaterial with a reduced number of required reactive components The invention relates to a powdered composition for preparing the inventive chemically bonded ceramic biomaterial, and a paste from which the biomaterial is formed, as well as a kit comprising the powdered composition and hydration liquid, as well as methods and use of the biomaterial in dental and implant applications.

### TECHNICAL FIELD

The present invention generally relates to chemically bonded ceramic biomaterials, preferably a dental material or an implant material, comprising two binder systems. The main binder system forms a chemically bonded ceramic upon hydration thereof, and comprises powdered calcium aluminate and, optionally, minor amounts of calcium silicate. The second binder system is a cross-linking organic binder system which provides for initial crosslinking of the freshly mixed paste forming the biomaterial. The biomaterial also comprises inert filler particles. The inventive biomaterial is free from reactive glass and thus provides for a simplified two binder systems biomaterial with a reduced number of required reactive components. The invention relates to a powdered composition for preparing the inventive chemically bonded ceramic biomaterial, and a paste from which the biomaterial is formed, as well as a kit comprising the powdered composition and hydration liquid, as well as methods and use of the biomaterial in dental and implant applications.

### STATE OF THE ART AND PROBLEM

In the art chemically bonded ceramic (CBC) biomaterials are known and have been described in a number of patent applications. Such materials are especially used in dental applications. A number of requirements should preferably be fulfilled by such materials. The material should be biocompatible with respect to its indication. Other properties of the biomaterial that are required especially for dental applications include good handling ability of the material with simple applicability in a cavity, moulding that permits good shaping ability, a hardening/solidification of the material that is sufficiently rapid for filling work without detrimental heat generation and that provides serviceability directly following therapy, a high hardness and strength, corrosion resistance of the resulting hardened material, good bonding between the hardened biomaterial and biological tissue, radio-opacity, good long time properties and good aesthetics of the resulting hardened material. The biomaterials may also comprise one or more additives, such as expansion compensating additives adapted to give the ceramic material dimensionally stable long-term attributes. For dental filling materials it is preferred that the system comprises additives and/or is based on raw materials that contribute to trans-lucency of the hydrated material.

WO 2005/039508 discloses a CBC system for dental and orthopaedic applications, which system has been developed to provide improved early-age properties and improved end-product properties, including bioactivity. The system includes two binding systems, a first initial working part-system, and second main system. The systems interact chemically. The main system is a cement-based system that comprises one or more CBCs selected from the group consisting of aluminates, silicates, phosphates, carbonates, sulphates and combinations thereof, having calcium as the major cation. The first binding system is based on a polycarboxylic acid, a co-polymer thereof, or a polycarboxylate (i.e. a salt or ester of a polycarboxylic acid), such as a polyacrylic acid and/or a salt thereof. The first binding system also requires the presence of an active glass for proper cross-linking thereof, and thus for obtaining the desired early-age properties of the overall system. For an optimised formation of the two part composite the CBC system requires Ca-aluminate or Ca-silicate, reactive glass, a poly acrylic acid and/or a salt thereof and inert filler particles.

According to the teachings of WO 2005/039508 glass ionomer cements consist of glass and poly acrylic acid. The acid dissolves the glass, and the ions from the glass cross-link the acid, and the material hardens. The reaction is rather rapid and nearly final strength is reached after about one hour.

The present inventors have surprisingly found that the system disclosed in WO 2005/039508 can be substantially simplified, while retaining the desired properties thereof. According to the present finding, inert filler particles, such as a stable glass can be used instead of the soluble (i.e. reactive) glass in the powder and composition disclosed in WO 2005/039508.

The invention has been especially developed for biomaterials for dental applications, preferably as a dental luting cement or restorative filling material, including fissure sealants, and dental cements for veneers.

### SUMMARY OF THE INVENTION

The present invention relates to biomaterials based on two binding systems, a first binder system of hydrating inorganic cement, and a second, cross-linking organic binder system, which biomaterial may be formed in situ, in vivo.

The present invention is based on the surprising finding that the soluble glass in the glass ionomer binding system of the biomaterial disclosed in WO 2005/039508 can be replaced by a stable glass. Thereby, a simplified system can be provided, having a reduced number of different required constituents, and thus allowing for a closer control and enhanced optimization of desired properties in the paste and resulting cured biomaterial. The material is especially intended for use as dental sealing material.

In one aspect the present invention relates to a powdered composition for preparing a chemically bonded ceramic biomaterial, which powdered composition comprises a powdered inorganic cement, which cement comprises calcium aluminate and, optionally, minor amounts of calcium silicate, a poly acrylic acid, and a stable, inert glass with a mean particle size of less than 2 μm.

In another aspect the invention relates to a paste obtained by mixing the powder composition with an aqueous hydration liquid based on water.

In a further aspect the invention relates to a kit comprising the powder and an aqueous hydration liquid based on water.

In another aspect the invention relates to a capsule mixing system containing the powder and an aqueous hydration liquid based on water.

In yet a further aspect the invention relates to a method of sealing an implant to another implant and/or to tooth or bone tissue using the paste of the invention.

In another aspect the invention relates to a method of cementing a veneer to a tooth using the paste of the invention.

The present invention addresses the issues for biomaterials based on chemically bonded ceramics for dental applications where sealing of the contact zone between the biomaterial and biological tissue is crucial, and wherein optimized early age properties as well as final properties are maintained. By using the material of the present invention, large un-dissolved glass particles in the contact zone, which may otherwise result from the material of WO 2005/039508, will be avoided. The avoidance of large un-dissolved glass particles is also of great importance when the material is used in the form of thin layers, such as dental cements and sealing materials to veneers.

### DETAILED DESCRIPTION OF THE INVENTION

The present invention is based on the surprising finding that the soluble glass in the glass ionomer binding system of the biomaterial disclosed in WO 2005/039508 can be replaced by a inert filler particles, such as a stable glass, and more particularly that calcium ions from the chemically bonded ceramic system both initially act as the active cation both in the cross-linking, and in the on-going hydration of the main system, i.e. the chemically bonded ceramic system. It has been found that, when a glass ionomer system used, such as in WO 2005/039508, the soluble glass is only to a limited extent dissolved, while the remaining soluble glass acts as a filler particle. The finding that the Ca-ions involved in the cross-linking polymerization are rather derived from the inorganic cement binder system than from the soluble glass in WO 2005/039508, means that the intended function of the soluble glass in WO 2005/039508, actually accomplished by the calcium cement, and makes the inclusion of a soluble glass according to WO 2005/039508 not an optimized solution. Surprisingly, in order to meet the specific requirements for the specific use of the biomaterial as a dental sealing material, a stable glass system is advantageously used instead. The use of a stable glass in such dental application allows for obtaining enhanced, optimized properties of the material, especially with regard to the end properties thereof.

The invention is aimed at producing biomaterials for dental applications with special reference to properties related to the microstructural development in the inventive material upon hydration thereof. The invention is described in more detail below.

### The Chemically Bonded Ceramic System.

The chemically bonded ceramic system of the invention is based on calcium aluminate. The preferred calcium aluminate phases are CA and/or C₁₂A₇. The mean average particle size should be below 5 μm, preferably below 4 μm, but not below 2 μm.

The initial low pH of the system due to the presence of a polycarboxylic acid, a copolymer thereof, or a polycarboxylate (i.e. a salt or ester of a polycarboxylic acid), such as polyacrylic acid and/or a salt thereof, induces a rapid dissolution of the basic calcium aluminate system. According to the invention also a low content (<10% by weight of the chemically bonding system, i.e. of the cement) of calcium silicate (C₂S and/or C₃S) can be included. These calcium silicate phases have an even more rapid dissolution than the calcium aluminate system.

The chemically bonded ceramics react with water, ions are formed, and hydrates are precipitated repeatedly. The particle size of the hydrates formed is below 100 nm.

### The Cross-Linking System.

According to the invention, the powdered material and/or the hydration liquid comprises a polycarboxylic acid (or a co-polymer thereof, or a polycarboxylate, i.e. a salt or ester of a polycarboxylic acid), such as e.g. a polyacrylic acid and/or a salt thereof. The polycarboxylic acid can be applied as a solution and/or as solid acid component.

The polycarboxylic acid may e.g. be selected from poly(maleic acid), poly(itaconic acid) or tricarballylic acid) or carboxylates thereof, such as phosphate esters.

Suitably, the polycarboxylic has a molecular weight of preferably 5,000-100,000 and is present in an amount of up to 30%, preferably 5-20%, and most preferably 10-15% by weight, calculated on the powdered material including any dry additives, such as e.g. used for dental applications. Preferably, the polycarboxylic has a molecular weight within the interval of 10,000-100,000.

### The Hydration Liquid

The bulk of the liquid used is water. The initial solution should have a pH<7 in order to enhance the dissolution of calcium aluminate, and any calcium silicate present, generating Ca-ions, which in turn will enhance the cross-linking of the polycarboxylic acid. The pH can according to the present invention preferably be higher than 5 in contrast to pure glass ionomer systems since the solubility of calcium aluminate, and any calcium silicate present is more pronounced. After cross-linking of the polycarboxylic acid, the pH is increased to over 8, at which pH the final hydration of the calcium aluminate, and any calcium silicate present, occurs. The control of the pH is essential for transforming the initial acid system into a bioactive system, i.e. for reaching conditions for apatite formation. The rapid change into high pH-values according to the present invention reduces the risk of free metal ion release, which is enhanced by acid conditions.

The amount of water is selected high enough according to the invention with the purpose of fully hydrating all the chemically bonding inorganic cement, so as to thereby enable the forming the chemically bonded ceramic. Thus, no or low contents of the original cement particles will remain in the end product.

### The Inert Filler Particles

As examples of the inert filler particles used according of the invention, stable glasses and/or oxides and/or pre-hydrated chemically bonded ceramics, such as dried calcium aluminate hydrates (i.e. CAH) can be mentioned. The chemically bonded ceramics are preferably CAH having a solid solution of heavy elements, which elements have a density of >5 g/cm³. Other possibilities according to the invention is to use micro-silica and/or nano-scale single crystal of hydrates, e.g. attapulgite, a magnesium silicate hydrate.

### Additives for Optimized Physical Properties

Inert, stable glass additives, preferably nano-size particles, may be included to achieve improved physical properties for both initial paste and the cured material. According to the present invention stable glass will contribute to improved properties of the early stage of the reacting material with in terms of rheology, including viscosity and cohesiveness. The improved properties of the cured material are specifically related to strength, wear resistance and fracture toughness, and optical properties. It is preferred that the system comprises inert nanosize glass, as an inert filler in the powdered material at high content, preferably a content corresponding to 40-65% by volume of the overall powder. The particle size is critical in establishing high homogeneity and related strength development. It is preferred that the particle size is 0.1-0.4 μm. The particle size is selected specifically with regard to the desired translucency of the resulting cured material, where the particle size in the preferred size range is below the lower region of the visual light, i.e. 400 nm. The inert glass particle composition should be comprised of glasses containing the element Sr and/or Ba and/or Zr, or other heavy element with a density >5 g/cm³.

### The Early and Final Curing in Detail

The system and materials according to the invention have the advantages over systems/materials, such as glass ionomer cements, and pure calcium aluminate based systems, and monomer based filling materials with regard to the combination of properties, in that the present system and material maintain their bioactivity, and in that they have improved initial strength, and in that they have long term stability in terms of both dimensional aspects, strength and minimized deterioration and a translucency >25%. The viscosity of the material can be controlled within wide ranges, upon initial mixing of the powdered material and the hydration liquid, from moist granules to an injectable slurry. The material reacts in two steps, i.e. by cross-linking of the organic acid and hydration of the inorganic calcium aluminate based binder system. Thereby, optimized early as well as optimized end-product properties are achieved.

The present invention may be used as a dental luting cement, tooth fillings, fissure sealings, and as endo products (including orthograde and retrograde fillings). The present invention is preferably used for sealing and related applications, such as dental luting cements, dental fillings including endodontic fillings, and cements for veneers.

### EXAMPLES

### Description of Raw Materials and Preparation

a. The calcium aluminate (CA═(CaO)(Al₂O₃)) used was synthesised and treated according to the description below.
b. Deionised water. (The water should be treated so that the main part of its ion content has been removed). The water could also preferably be further treated in order to remove microorganisms and other impurities).
c. Poly acrylic acid, p.a. quality, having an average molecular weight in the interval of 10,000-100,000.
d. Inert glass of the composition SiO₂—BaO—B₂O₃—Al₂O₃ in wt % 50-30-10-10 average particle size 0.4 μm, d(99)<3 μm.
e. LiCl was used either as crystals or pre-prepared standard solutions, p.a. quality.
f. Neutralised sodium Nitrilo Tri acetic Acid (Na₃—NTA), either as crystals, powders or pre-prepared standard solutions were used.

### Example 1

### Preparation of the Powder

The calcium aluminate used for this material was synthesised using high purity Al₂O₃ and either of CaO and CaCO₃. The correct amounts of the raw materials are weighed in to a suitable container (1:1 molar ratio). The powders are intimately mixed by tumbling in excess isopropanol or tumbled dry using a dry powder mixer. If mixing in isopropanol is performed the next step will be removing the isopropanol, such as by evaporation of the solvent using an evaporator combining vacuum and heat and finally heating in oven. The next step is filling high purity Al₂O₃ crucibles with the powder mix and heat treating it above 1350° C. for the appropriate amount of time in order to get nearly mono phase calcium aluminate according to the description above. After heat treatment the material is crushed using a high energy crusher, in this case a roller crusher with alumina rollers. After crushing the calcium aluminate is milled using an air jet mill (Hosokawa Alpine) to the specified particle size distribution with a d(99)v of 10 μm <d(99)v<12 μm and an average particle size of 4 μm.

The final powder formulation is obtained in the following way: All powder components are weighed in with high accuracy according to the composition in Table 1.

TABLE 1
Composition of the final powder formulation.

| Raw material | Wt % |
|---|---|
| Calcium aluminate | 40.0 |
| CA phase | |
| Polyacrylic acid | 10.50 |
| Tartaric acid | 2.00 |
| Glass (inert) | 47.50 |
| Ps < 0.5 μm | |

The components are weighed into a glass beaker, and the beaker is thereafter placed in a dry mixer and the components mixed at medium speed for 3 hours. The next step after mixing is sieving through a 125 μm sieve in order to homogenise the powder and remove large agglomerates. After sieving, the powder is transferred to a suitable container, which is then sealed and stored dry. The powder is now ready for use.

### Example 2

### Preparation of the Liquid

The LiCl is first dried at 150° C. for at least 2 hours in order to remove physically bound water. The LiCl is weighed into a PE bottle so that the final composition after addition of the water will be 25 mM of LiCl and 0.35 wt % of Na₃-NTA. After the water has been added the bottle is shaken until all the salts have dissolved. The liquid is now ready for use.

### Example 3

### Description of Tests and Results Based on Standard Tests

The powder and liquid described above were tested together in the below tests using a powder to liquid (P:L) ratio of 3.0:1.0. The material is mixed by hand using a spatula by bringing the required amount of powder and liquid on to a mixing pad and mixing them thoroughly for 35 seconds. A capsule system can also be used. In the latter case the powder and liquid are pre-filled in the correct amounts to generate the required P:L ratio=3.0:1.0, into a dental capsule system. The capsule is then transferred to a capsule mixing machine and mixed for a sufficient period of time. Using a 3M/ESPE Rotomix the time should be >6 s with a 3 s centrifuge stage in the end. After mixing, the ready material is dispensed using a conventional applicator into any desired sample mould or container. There is no significant difference in properties depending on whether the material is well-mixed by hand, or using a capsule system.

The tests performed on the material are the tests shown in Table 2.

TABLE 2

| Test | Controlling standard |
|---|---|
| Net setting time | ISO 9917:2003 part 1 |
| Film thickness | ISO 9917:2003 part 1 |
| Compressive strength | ISO 9917:2003 part 1 |
| Acid erosion | ISO 9917:2003 part 1 |
| Radio Opacity | ISO 9917:2003 part 2 |
| Translucency/Opacity | ISO 4049 |
| In vitro bioactivity | Formation of apatite |

The results show that by producing a sealing material according to the above description and using it with a P:L ratio of 3.0:1.0 all the above tests according to ISO 9917:2003 were fulfilled.

Regarding the bioactivity, it has been shown by means of energy dispersive spectroscopy (EDS), scanning electron microscopy (SEM), transmission electron microscopy (TEM), grazing incidence X-ray diffraction (GI-XRD) that a layer of crystallised hydroxyl apatite is formed on the surface of the material when submerged in phosphate buffered saline (PBS) for a period of 2-30 days.

### Example 3

Fracture toughness was measured using the single edged notch technique, and was determined to be 0.7 MPam^{1/2}.

The invention is not limited to the embodiments described, but can be varied within the scope of the claims.

1. A powdered composition for preparing a chemically bonded ceramic biomaterial comprising: a powdered inorganic cement comprising calcium aluminate; a polycarboxylic acid; a co-polymer thereof, or a polycarboxylate; and inert filler particles, characterised in that the filler particles have a mean particle size of less than 2 μm, no reactive glass is present, and in that the polycarboxylic acid, co-polymer thereof, or polycarboxylate has a molecular weight of 5,000-100,000 and is present in an amount of 5-30% by weight, calculated based on the powdered material including any dry additives.

2. The composition of claim 1, wherein the mean particle size of the inert filler particles is within the range of 0.1-0.4 μm.

3. The composition of claim 1, wherein the mean particle size of the calcium aluminate is below 5 μm.

4. The composition of claim 1, wherein the inert filler particles comprise pre-hydrated, dried CAH phases having a solid solution of elements having a density of >5 g/cm³.

5. A paste obtainable by combining:
a powdered inorganic cement comprising calcium aluminate;
a polycarboxylic acid, a co-polymer thereof, or a polycarboxylate;
inert filler particles; and
an aqueous hydration liquid based on water,
characterised in that the filler particles have a mean particle size of less than 2 μm, no reactive glass is included, and in that the polycarboxylic acid, co-polymer thereof, or polycarboxylate has a molecular weight of 5,000-100,000 and is present in an amount of 5-30% by weight, calculated based on the powdered material including any dry additives.

6. The paste of claim 5, wherein the mean particle size of the inert filler particles is within the range of 0.1-0.4 μm.

7. The paste of claim 5, wherein the mean particle size of the calcium aluminate is below 5 μm.

8. A kit comprising the powder of claim 1 and an aqueous hydration liquid based on water.

9. The kit of claim 8 in the form of a capsule mixing system containing the powder of claim 1 and an aqueous hydration liquid based on water.

10-12. (canceled)

13. A method of sealing an implant to another implant and/or to tooth or bone tissue wherein the paste of claim 5 is used.

14. A method of cementing a veneer to a tooth wherein the paste of claim 5 is used.
